(19) United States
(12) Patent Application Publication
(10) Pub. No.: US 2015/0368316 A1
(43) Pub. Date: Dec. 24, 2015

(54) A SELECTIVE HIGH-AFFINITY IMMUNE STIMULATORY REAGENT AND USES THEREOF

(71) Applicant: ALBERT EINSTEIN COLLEGE OF MEDICINE OF YESHIVA UNIVERSITY, Bronx, NY (US)

(72) Inventors: Eszter LAZAR-MOLNAR, Salt Lake City, UT (US); Steven C. ALMO, Pelham, NY (US)

(73) Assignee: ALBERT EINSTEIN COLLEGE OF MEDICINE OF YESHIVA UNIVERSITY, Bronx, NY (US)

(21) Appl. No.: 14/765,885

(22) PCT Filed: Feb. 7, 2014
(86) PCT No.: PCT/US2014/015235

§ 371 Date: Aug. 5, 2015

Related U.S. Application Data
(60) Provisional application No. 61/761,755, filed on Feb. 7, 2013.

(51) Int. Cl. C07K 14/705

(52) U.S. Cl. CPC C07K 14/70503; C07K 2319/30; A61K 38/00

(57) ABSTRACT

Isolated polypeptides comprising a mutant programmed cell death 1 (PD-1) polypeptide and fusion polypeptides comprising an isolated mutant PD-1 polypeptide fused to an immunoglobulin domain polypeptide are provided. Further provided are methods of using a composition comprising the fusion polypeptide for stimulating T cell activation for treating disorders including a tumor or an infection.

### CROSS-REFERENCE TO RELATED APPLICATIONS

This application claims benefit of U.S. Provisional Application No. 61/761,755, filed Feb. 7, 2013, the contents of which are hereby incorporated by reference.

### STATEMENT OF GOVERNMENT SUPPORT

This invention was made with government support under grant numbers 5U54GM094662, 5U01GM094665, and 5R01AI007289, awarded by the National Institutes of Health. The government has certain rights in this invention.

### BACKGROUND OF THE INVENTION

Throughout this application various publications are referred to. Full citations for these references may be found at the end of the specification. The disclosures of these publications, and of all patents, patent application publications and books referred to herein, are hereby incorporated by reference in their entirety into the subject application to more fully describe the art to which the subject invention pertains.

In recent years, T cell costimulatory pathways have been identified as versatile novel targets for immunotherapeutic strategies. The CD28:B7 family of costimulatory molecules includes CD28 and ICOS as positive co-receptors, and CTLA4 and PD-1 as co-inhibitors, which tightly regulate all T cell activation processes.

Enhancing T cell activation by blockade of the PD-L/PD-1 inhibitory pathway has enormous potential for the treatment of infectious diseases and malignant tumors. Recent studies have shown that enhancing T cell activation by blocking PD-1 could be beneficial in chronic viral infections, as well as other infections in which this costimulatory pathway is involved. Host responses to pathogens such as fungi, protozoa, worms and bacteria have been shown to be regulated by PD-1, and therefore could be improved by manipulating the PD-1 pathway.

Although targeting costimulatory pathways is a relatively recent approach, there are a number of antibodies approved for clinical use and myriad others in development for clinical trials. One such FDA-approved drug is Yervoy (Ipilimumab), which is a monoclonal antibody directed against the co-inhibitory receptor CTLA-4. Yervoy has been shown to be effective in increasing survival of metastatic melanoma patients (10 months median survival for the antibody treated group versus 6.4 months for the control group, Hodi F S et al, N Engl J Med 2010). Yervoy acts through inducing activation of T cells by blocking CTLA-4, causing significant immune stimulation, including anti-tumor immune responses. Due to the central role of CTLA-4 in all immune responses (central and peripheral), Yervoy can cause significant side effects associated with an overly active immune response, e.g. autoimmune symptoms can develop and in some cases these can be lethal. Notably, the absence of CTLA-4 in mice caused by genetic deletion is lethal, highlighting the importance of this molecular “break” on the general immune response.

The present invention addresses the need for improved targeting of costimulatory pathways by manipulating the PD-1 pathway and provides a high affinity PD-1-based immune stimulatory agent.

### SUMMARY OF THE INVENTION

This invention provides an isolated polypeptide comprising a mutant PD-1 polypeptide, wherein the mutant PD-1 polypeptide is a mutant by having an A132L mutation relative to SEQ ID NO:7 or to NCBI Reference Sequence NP_{—}005009.2.

This invention also provides a fusion polypeptide comprising the isolated polypeptide comprising a A132L mutant PD-1 polypeptide described herein, fused to an immunoglobulin domain polypeptide.

This invention also provides a mutant PD-1 comprising consecutive amino acid residues (i) having the sequence set forth in SEQ ID NO:4, or (ii) having a sequence 95% or greater identical to SEQ ID NO:4 and comprising an L at the residue corresponding to A132 relative to NCBI Reference Sequence NP_{—}005009.2.

This invention also provides a homo-oligomer comprising the isolated polypeptide comprising a A132L mutant PD-1 polypeptide, or comprising the fusion polypeptide. In a preferred embodiment, the homo-oligomer comprises two of the isolated polypeptides, or two of the fusion polypeptides.

This invention also provides a composition comprising the isolated polypeptide in monovalent form or oligomeric form. This invention also provides a composition comprising the fusion polypeptide in monovalent form or oligomeric form.

Also provided is a method of stimulating T cell activation in a subject comprising administering to the subject the isolated polypeptide described herein, or the composition or homo-oligomer comprising the isolated polypeptide described herein, in an amount sufficient to stimulate T cell activation in a subject.

Also provided is a method of stimulating T cell activation in a subject comprising administering to the subject the isolated fusion polypeptide described herein, or the composition or homo-oligomer comprising the isolated fusion polypeptide described herein, in an amount sufficient to stimulate T cell activation in a subject.

Also provided is a method of treating a tumor, or treating an infection in a subject comprising administering to the subject the isolated fusion polypeptide described herein, or the composition or homo-oligomer comprising the isolated fusion polypeptide described herein, in an amount sufficient to stimulate T cell activation, treat a tumor, or treat an infection, respectively, in a subject.

### BRIEF DESCRIPTION OF THE DRAWINGS

FIG. 1A-1D. Binding affinities of wild-type and A132 mutant PD-1 to PD-L1 and PD-L2. SPR data and equilibrium dissociation constants between immobilized human PD-L1 and A) wild-type, or B) A132 mutant soluble PD-1, as well as immobilized human PD-L2 and C) wild-type or D) A132L mutant PD-1. K_{d}s are shown in μM, standard errors from data fitting are shown.

FIG. 2A-2B. Human high-affinity PD-1 Ig (HA PD-1 Ig) shows increased binding to human monocyte-derived dendritic cells expressing PD-L1 and PD-L2. Monocytes were obtained from PBMCs of healthy donors and differentiated into dendritic cells over 5 days in the presence of GM-CSF and IL-4, then treated with TNF-α to promote the formation of “mature” dendritic cells. A) After TNF-α treatment, PD-L1 and PD-L2 are upregulated on the surface of DCs. Blue histograms: PD-L1 or PD-L2 antibody staining; red histograms: isotype controls. B) Increased binding of HA PD-1 Ig to “mature” human DCs. K_{d} value is calculated from the mean fluorescence intensities (MFI) of Ig fusion protein binding, as detected by flow cytometry. Although K_{d} values are estimates, the HA PD-1 Ig exhibits considerably increased avidity compared to the wild type.

FIG. 3. High-affinity PD-1 Ig increases T cell proliferation in allogeneic mixed lymphocyte reaction (MLR) assay. Dendritic cells differentiated as described for FIG. 2 were co-cultured with human T cells derived from an allogeneic donor (16:1 T cell:DC ratio), in the presence of high-affinity versus wild-type PD-1 Ig, or control proteins. 3H-thymidine was added for the last 18 hours of culture, and cell proliferation was determined after 5 days of culture. Monoclonal PD-L1 blocking antibody was used as positive control, IgG1 as negative control. All proteins were used at 5 μg/ml concentration, except HA PD-1 Ig, which was titrated from 0.05 to 5 μg/ml. Error bars represent standard errors of mean for replicates of three wells. Two-way ANOVA followed by Bonferoni post-test was used to analyze the data, **** indicates p<0.0001.

FIG. 4A-4B. High affinity PD-1 Ig increases T cell cytokine secretion in allogeneic mixed lymphocyte reaction (MLR) assay. Dendritic cells differentiated as described for FIG. 2 were co-cultured with human T cells from an allogeneic donor (16:1 T cell:DC ratio), in the presence of high-affinity versus wild-type PD-1 Ig, or control proteins. PD-L1 blocking antibody was used as positive, and IgG1 as a negative control; all proteins were used at 5 μg/ml concentration. Cell culture supernatants were collected from 4-day cultures, and cytokines were measured using 11plex FlowCytomix kit from eBioscience. Error bars represent standard errors of mean for replicates of three. One-way ANOVA followed by Dunnett's multiple comparison post-test was used to analyze the data, Statistical significance is shown between experimental groups compared to the control untreated (No Ig) group, * indicated p, 0.05, *** indicates p<0.0005.

### DETAILED DESCRIPTION OF THE INVENTION

This invention also provides an isolated mutant PD-1 polypeptide, wherein the mutant PD-1 polypeptide is a mutant by having an A132L mutation relative to SEQ ID NO:7 or to NCBI Reference Sequence NP_{—}005009.2. In an embodiment, the mutant PD-1 comprises consecutive amino acid residues having the sequence set forth in SEQ ID NO:4.

An isolated mutant PD-1 polypeptide, wherein the mutant PD-1 polypeptide is a mutant by having an A132L mutation relative to the PD-1 polypeptide in SEQ ID NO:7 or an A132L mutation relative to PD-1 polypeptide in NCBI Reference Sequence NP_{—}005009.2. In an embodiment, the polypeptide is in monovalent form. In an embodiment, the mutant polypeptide is soluble. In an embodiment, the mutant polypeptide does not comprise a transmembrane domain. In an embodiment, the mutant polypeptide does not comprise a intracellular domain. In an embodiment, the mutant polypeptide comprises a sequence having the same sequence as a PD-1 transmembrane domain. In an embodiment, the mutant polypeptide comprises a sequence having the same sequence as a PD-1 intracellular domain.

In an embodiment, the mutant PD-1 polypeptide comprises consecutive amino acid residues (i) having the sequence set forth in SEQ ID NO:4, or (ii) having a sequence 95% or greater identical to SEQ ID NO:4 and comprising an L at the residue corresponding to A132 relative to NCBI Reference Sequence NP_{—}005009.2. In an embodiment, the mutant PD-1 polypeptide comprises consecutive amino acid residues having a sequence 96% or greater identical to SEQ ID NO:4. In an embodiment, the mutant PD-1 polypeptide comprises consecutive amino acid residues having a sequence 97% or greater identical to SEQ ID NO:4. In an embodiment, the mutant PD-1 polypeptide comprises consecutive amino acid residues having a sequence 98% or greater identical to SEQ ID NO:4. In an embodiment, the mutant PD-1 polypeptide comprises consecutive amino acid residues having a sequence 99% or greater identical to SEQ ID NO:4.

It is noted that one skilled in the art can determine which residue of any PD-1 variant corresponds to the A132 residue identified herein and that can be mutated to an L, e.g. by simple majority alignment or other methods.

Substitution variants of the mutant PD-1, as provided by the invention, have at least one amino acid residue in the polypeptide removed and a different residue inserted in its place (except for the A132 or equivalent residue that has been mutated to an L). In an embodiment, the substitution is a conservative substitution. Conservative substitutions are shown in Table 1 under the heading of “conservative substitutions.” In an embodiment, the substitution is an exemplary substitution as listed in Table 1. In an embodiment, the PD-1 mutant contains one of 1, 2, 3, 4 or 5 substitutions relative to SEQ ID NO: 7, with or without the residues 1-20 signal peptide.

TABLE 1
Amino Acid Substitutions

| Original | | |
|---|---|---|
| Residue | Conservative Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

This invention also provides a fusion polypeptide comprising the isolated polypeptide comprising a A132L mutant PD-1 polypeptide described herein, fused to an immunoglobulin domain polypeptide. In an embodiment, the mutant PD-1 polypeptide is fused to the immunoglobulin domain polypeptide by a peptide bond between a terminal amino acid of the mutant PD-1 polypeptide and a terminal amino acid of the immunoglobulin domain polypeptide. In an embodiment, the immunoglobulin domain polypeptide comprises an immunoglobulin IgG Fc domain. In an embodiment, the immunoglobulin domain polypeptide comprises an immunoglobulin IgM Fc domain. In an embodiment, the immunoglobulin domain polypeptide comprises an immunoglobulin IgG1 Fc domain. In an embodiment, the immunoglobulin IgG or IgM Fc domain is human. In an embodiment, the immunoglobulin IgG1 Fc domain is human. In an embodiment, the fusion polypeptide is in monovalent form. In an embodiment, the fusion polypeptide comprises SEQ ID NO:2 as disclosed herein.

This invention also provides a homo-oligomer comprising the isolated polypeptide comprising a A132L mutant PD-1 polypeptide, or comprising the fusion polypeptide. In a preferred embodiment, the homo-oligomer comprises two of the isolated polypeptides, or two of the fusion polypeptides.

This invention also provides a composition comprising the isolated polypeptide in monovalent form or oligomeric form. This invention also provides a composition comprising the fusion polypeptide in monovalent form or oligomeric form. In an embodiment, the compositions comprise a pharmaceutically acceptable carrier. In an embodiment, the pharmaceutically acceptable carrier comprises a single type of molecule. In an embodiment, the pharmaceutically acceptable carrier comprises a mixture of molecules.

Also provided is a method of stimulating T cell activation in a subject comprising administering to the subject the isolated polypeptide described herein, or the composition or homo-oligomer comprising the isolated polypeptide described herein, in an amount sufficient to stimulate T cell activation in a subject. In an embodiment, the subject has a tumor. In an embodiment, the subject has an infection.

Also provided is a method of treating a tumor in a subject comprising administering to the subject the isolated fusion polypeptide described herein, or the composition or homo-oligomer comprising the isolated fusion polypeptide described herein, in an amount sufficient to treat a tumor in a subject.

In an embodiment, the tumor is a tumor of the breast, lung, colon, ovarian, melanoma, bladder, liver, salivary, stomach, gliomas, thyroid, thymus, epithelial, head, or neck. In an embodiment, the tumor is a hematological malignancy. In an embodiment, the tumor is a lymphoma. In an embodiment, the tumor is a myeloma. In an embodiment, the tumor is a multiple myeloma.

Also provided is a method of treating an infection in a subject comprising administering to the subject the isolated fusion polypeptide described herein, or the composition or homo-oligomer comprising the isolated fusion polypeptide described herein, in an amount sufficient to treat an infection in a subject.

In an embodiment, the infection is a viral Infection. In a further embodiment, the virus is a HIV, HCV, HBV or HTLV. In an embodiment, the infection is a bacterial, fungal, protozoal or parasitic infection. In embodiments, the infection is caused by Helicobacter pylori, the fungus Histoplasma capsulatum, the parasite Taenia crassiceps or Schistosoma mansoni, or the protozoa Leishmania mexicana.

In an embodiment, the mutant PD-1 comprises SEQ ID NO:3 as disclosed herein. In an embodiment, the mutant PD-1 comprises SEQ ID NO:3 as disclosed herein without the first 20 amino acid residues as counted from the N-terminal. In an embodiment, the mutant PD-1 comprises SEQ ID NO:4 as disclosed herein. In an embodiment, the mutant PD-1 comprises SEQ ID NO:5 as disclosed herein. In an embodiment, the mutant PD-1 comprises SEQ ID NO:6 as disclosed herein. In an embodiment, the mutant PD-1 comprises SEQ ID NO:6 as disclosed herein without the first 20 amino acid residues as counted from the N-terminal

(SEQ ID NO: 3)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKLQIKESLRAELRVTERRAE
VPTAHPSPSPRPAGQFQ;
(SEQ ID NO: 4)
PGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKLQIKESLRAELRVTERRAE
VPTAHPSPSPRPAGQFQ;
(SEQ ID NO: 5)
PGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKLQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL;
and
(SEQ ID NO: 6)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKLQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

In an embodiment, the un-mutated full-length PD-1 has the following sequence:

(SEQ ID NO: 7)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL

In an embodiment, the mutant is a mutant relative to NCBI Reference Sequence NP_{—}005009.2.

In an embodiment, the composition is a pharmaceutical composition. In an embodiment, the pharmaceutical composition comprises a pharmaceutically acceptable carrier. As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

Also provided is an isolated nucleic acid encoding an isolated mutant PD-1 polypeptide described herein. Also provided is an isolated nucleic acid encoding a fusion polypeptide described herein. In an embodiment, the nucleic acid a recombinant nucleic acid. In an embodiment, the nucleic acid is an RNA. In an embodiment, the nucleic acid is an DNA. In an embodiment, the nucleic acid comprises cDNA.

An isolated cell containing a vector comprising a nucleic acid encoding an isolated mutant PD-1 polypeptide described herein is also provided. In an embodiment, the cell I used for production of the mutant PD-1 polypeptide.

As described herein, a mutant PD-1 polypeptide is a not a naturally occurring mutant PD-1 polypeptide.

Also provided is an isolated mutant PD-1 polypeptide, as described hereinabove, wherein the mutant PD-1 polypeptide is a mutant by having an A132L mutation relative to SEQ ID NO:7 or an A132L mutation relative to NCBI Reference Sequence NP_{—}005009.2, or a fusion polypeptide comprising the polypeptide, as described hereinabove, fused to an immunoglobulin domain polypeptide, for treating an infection in a subject, or for treating a tumor in a subject, or for stimulating T-cell activation in a subject. In an embodiment, the mutant PD-1 polypeptide is for treating an infection in a subject. Exemplary infections are described hereinabove. n an embodiment, the mutant PD-1 polypeptide is for treating a tumor in a subject. Exemplary tumors are described hereinabove. n an embodiment, the mutant PD-1 polypeptide is for stimulating T-cell activation in a subject.

In a preferred embodiment of the methods, the subject is a human.

Also provided is a composition comprising a dendritic cell, loaded with any of the isolated mutant polypeptides or fusion proteins described herein. In an embodiment, the dendritic cell is mammalian. In an embodiment, it is derived from a human. In an embodiment, it is not derived from a human.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

In recent years, the targeting of T cell costimulatory pathways has been demonstrated to represent powerful and effective strategies for immunotherapy. The best characterized costimulatory pathways include those associated with members of the CD28:B7 family, such as CD28 and ICOS as positive co-receptors, and CTLA4 and PD-1 as co-inhibitors. Herein, structure-guided approaches have been used to develop a novel protein reagent by site-directed mutagenesis that targets the PD-1 pathway.

### Results

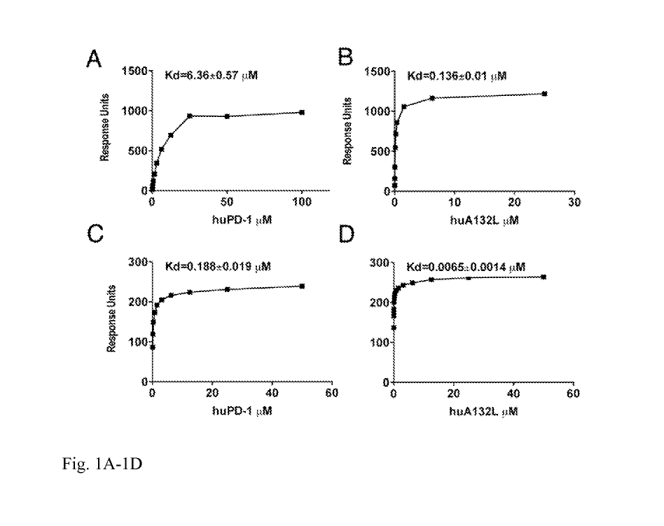

The crystal structure of human PD-1 was determined and a human PD-1 receptor mutant developed, which in its monovalent form exhibits over 50- and 30-fold higher affinity to its two ligands, PD-L1 and PD-L2, respectively, compared to wild-type PD-1 (Table 1 and FIG. 1). The A132 mutant PD-1 was made and the binding affinities of wild-type and A132 mutant PD-1 to PD-L1 and PD-L2 were compared. FIG. 1 shows SPR data and equilibrium dissociation constants between immobilized human PD-L1 and 1A) wild-type, or 1B) A132 mutant soluble PD-1, as well as immobilized human PD-L2 and 1C) wild-type or D) A132L mutant PD-1. Also see Table 2.

HA huPD-1-Ig DNA sequence of the full length expression construct:

BglII r. site + Kozak + beta-2m-signal
peptide (20aa) + huPD-1 (P21-Q167) +
SalI + thrombin + linker + huIgG1 Fc
(Pro100-Lys330) + hexaHis + STOP + BamHI
(SEQ ID NO: 1)
AGATCTgccgccaccATGTCTCGCTCCGTGGCCTTAGCTGTGCTCGCGCT
ACTCTCTCTTTCTGGCCTGGAGGCTccaggatggttcttagactccccag
acaggccctggaacccccccaccttctccccagccctgctcgtggtgacc
gaaggggacaacgccaccttcacctgcagcttctccaacacatcggagag
cttcgtgctaaactggtaccgcatgagccccagcaaccagacggacaagc
tggccgccttccccgaggaccgcagccagcccggccaggactgccgcttc
cgtgtcacacaactgcccaacgggcgtgacttccacatgagcgtggtcag
ggcccggcgcaatgacagcggcacctacctctgtggggccatctccctgg
cccccaagCTGcagatcaaagagagcctgcgggcagagctcagggtgaca
gagagaagggcagaagtgcccacagcccaccccagcccctcacccaggcc
agccggccagttccaaGTCGACttggtccctcgtggtagtGGAGGCTCTc
ccaaatcttgtgacaaaactcacacatgcccaccgtgcccagcacctgaa
ctcctggggggaccgtcagtcttcctcttccccccaaaacccaaggacac
cctcatgatctcccggacccctgaggtcacatgcgtggtggtggacgtga
gccacgaagaccctgaggtcaagttcaactggtacgtggacggcgtggag
gtgcataatgccaagacaaagccgcgggaggagcagtacaacagcacgta
ccgtgtggtcagcgtcctcaccgtcctgcaccaggactggctgaatggca
aggagtacaagtgcaaggtctccaacaaagccctcccagcccccatcgag
aaaaccatctccaaagccaaagggcagccccgagaaccacaggtgtacac
cctgcccccatcccgggaggagatgaccaagaaccaggtcagcctgacct
gcctggtcaaaggcttctatcccagcgacatcgccgtggagtgggagagc
aatgggcagccggagaacaactacaagaccacgcctcccgtgctggactc
cgacggctccttcttcctctatagcaagctcaccgtggacaagagcaggt
ggcagcaggggaacgtcttctcatgctccgtgatgcatgaggctctgcac
aaccactacacgcagaagagcctctccctgtctccgggtaaaCATCATCA
CCATCACCATtgaGGATCC - 1269 bp total..

Position 409-411 in the construct DNA sequence (SEQ ID NO:1) contains a GCG to CTG mutation (underlined), resulting in Alanine to Leucine mutation at position 132 in the mature full length protein.

HA huPD-1-Ig protein sequence (SEQ ID NO: 2):
PGWFLDSPDRPWNPPTFSPALLVVTEGDNATFTCSFSNTSESFVLNWYRM
SPSNQTDKLAAFPEDRSQPGQDCRFRVTQLPNGRDFHMSVVRARRNDSGT
YLCGAISLAPKLQIKESLRAELRVTERRAEVPTAHPSPSPRSAGQFQVDL
VPRGSGGSPKSCDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH -
Length: 395 amino acid residues total.

At position 112 of the expressed HA PD-1 Ig (SEQ ID NO:2) the Alanine to Leucine mutation is shown underlined (which corresponds to position 132 of the mature full length protein).

TABLE 2
Equilibrium dissociation constants for human PD-1 and its high-affinity
(HA) A132L mutant to PD-L1 and PD-L2.

| | huPD-1 | huA132L |
|---|---|---|
| huPD-L1 | 6.355 ± 0.566 | 0.136 ± 0.010 |
| huPD-L2 | 0.188 ± 0.019 | 0.0065 ± 0.0014 |

Equilibrium dissociation constants (K_{d}s) were determined by non-linear fitting of SPR data, using immobilized PD-Ligands and soluble wild-type or mutant PD-1. K_{d} values are in μM concentration, followed by the standard errors of the fittings.

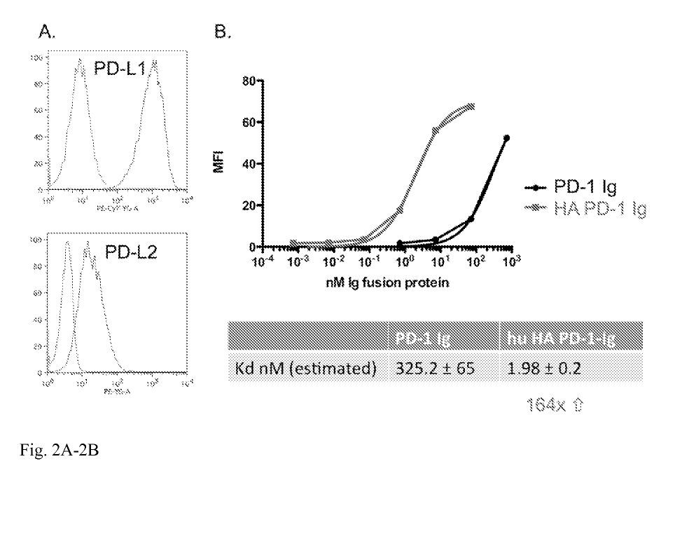

The fusion human high-affinity PD-1 Ig (HA PD-1 Ig) shows increased binding to human monocyte-derived dendritic cells expressing PD-L1 and PD-L2. As shown in FIG. 2, monocytes were obtained from PBMCs of healthy donors and differentiated into dendritic cells over 5 days in the presence of GM-CSF and IL-4, then treated with TNF-α to promote the formation of “mature” dendritic cells. FIG. 2A shows after TNF-α treatment, PD-L1 and PD-L2 are upregulated on the surface of DCs. Blue histograms: PD-L1 or PD-L2 antibody staining; red histograms: isotype controls. FIG. 2B) shows increased binding of HA PD-1 Ig to “mature” human DCs. K_{d} value is calculated from the mean fluorescence intensities (MFI) of Ig fusion protein binding, as detected by flow cytometry. Although K_{d} values are estimates, the HA PD-1 Ig exhibits considerably increased avidity compared to the wild type.

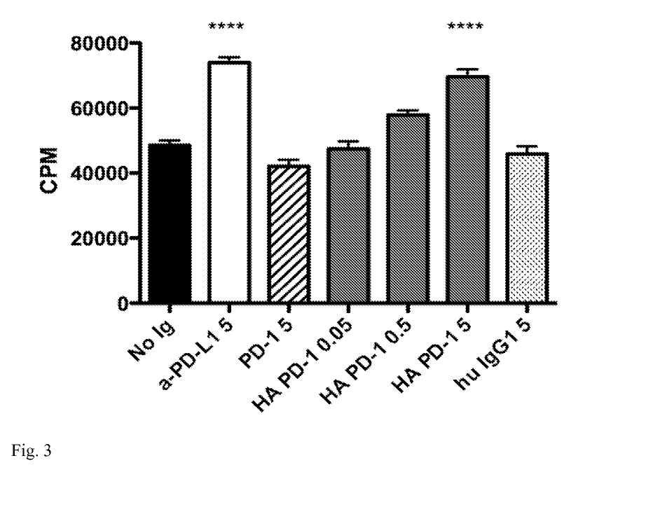

For use as a potent blocker of the endogenous PD-1/PD-L pathway, a dimeric soluble Fc chimera of this mutant was engineered by fusing it to the Fc part of human IgG1. Due to its higher avidity (>100-fold tighter binding than wild type), the soluble mutant PD-1 Ig reagent binds the PD-1 ligands in vitro or in vivo (FIG. 2), and prevents their binding to the endogenous PD-1 expressed on T cells, thus preventing inhibitory signals to these cells. High-affinity PD-1 Ig was found to increase T cell proliferation in allogeneic mixed lymphocyte reaction (MLR) assay. As shown in FIG. 3, dendritic cells differentiated as described for FIG. 2 were co-cultured with human T cells derived from an allogeneic donor (16:1 T cell:DC ratio), in the presence of high-affinity versus wild-type PD-1 Ig, or control proteins. ³H-thymidine was added for the last 18 hours of culture, and cell proliferation was determined after 5 days of culture. Monoclonal PD-L1 blocking antibody was used as positive control, IgG1 as negative control. All proteins were used at 5 μg/ml concentration, except HA PD-1 Ig, which was titrated from 0.05 to 5 μg/ml.

High affinity PD-1 Ig was also found to increase T cell cytokine secretion in allogeneic mixed lymphocyte reaction (MLR) assay. Dendritic cells differentiated as described for FIG. 2 were co-cultured with human T cells from an allogeneic donor (16:1 T cell:DC ratio), in the presence of high-affinity versus wild-type PD-1 Ig, or control proteins. PD-L1 blocking antibody was used as positive, and IgG1 as a negative control; all proteins were used at 5 μg/ml concentration. Cell culture supernatants were collected from 4-day cultures, and cytokines were measured using 11plex FlowCytomix kit from eBioscience. Error bars represent standard errors of mean for replicates of three.

TABLE 3
Human HA PD-1 mutant has increased affinity to mouse PD-L1 and
PD-L2

| | m PD-1 | m HA PD-1 | hu PD-1 | hu HA PD-1 |
|---|---|---|---|---|
| mPD- | 5.43 ± 0.08 | 2.78 ± 0.03 | 3.81 ± 0.08 | 0.23 ± 0.01 |
| L1-Ig | | | | |
| huPD- | 4.02 ± 0.06 | 0.48 ± 0.02 | 6.35 ± 0.57 | 0.14 ± 0.01 |
| L1-Ig | | | | |
| mPD- | 2.80 ± 0.15 | 0.47 ± 0.02 | 0.27 ± 0.01 | 0.029 ± 0.004 |
| L2-Ig | | | | |
| huPD- | 10.69 ± 0.83 | 1.18 ± 0.08 | 0.19 ± 0.02 | 0.0065 ± 0.0014 |
| L2-Ig | | | | |

Table 3 shows Kd values in μM concentration, followed by the standard errors of the fittings.

### Discussion

When presented in its monovalent form, the rationally designed mutant PD-1 receptor disclosed herein exhibits 50- and 30-fold higher affinity for both of its ligands, PD-L1 and PD-L2, respectively, compared to the wild-type receptor. When presented in the context of a bivalent Ig-fusion protein, this mutant exhibits greater than two orders of magnitude higher avidity for target T cells compared to the wild type PD-1 receptor. In addition to its enhanced binding properties, this mutant Ig-fusion construct elicited a range of potentially desirable cytokine responses distinct from those associated with blocking monoclonal antibodies. By selectively blocking an important T cell inhibitory pathway, the reagent represents a novel strategy to enhance T cell responses to infectious agents and malignancies, with reduced side effects compared to existing biologics such as Yervoy, the function blocking mAb targeting CTLA-4.

The mutant PD-1 receptor fusion protein can elicit immune stimulation by binding specifically to well-defined cell-surface targets that inhibit T cell-mediated immune responses against infections and malignancies. Importantly, the reagent specifically upregulates anti-pathogen and anti-tumor immune responses, as the PD-1 pathways are commonly used by pathogens and tumors for immune evasion. As an example: a variety of pathogens (viruses, bacteria, fungi, protozoa) manipulate the immune response after infection (immune evasion) for their own benefit. This is often accomplished by induction of inhibitory ligands (PD-L1 and PD-L2) on the surface of the infected cells, which in turn will bind to and provide inhibitory signals (through PD-1 pathways) to effector cells such as T cells needed for clearance of the infection. Similarly, tumor cells can upregulate these same inhibitory ligands to prevent immune attack from tumor-specific T cells. Since the reagent herein disclosed binds with high affinity to the PD-L1 and PD-L2 inhibitory ligands, it will impair the inhibitory signals used by infected cells and tumor cells to evade the immune response.

Blocking the PD-1/PD-L pathways using the high-affinity PD-1 Ig disclosed herein would result in an enhanced immune response, similar to that of the CTLA-4 blockade caused by Yervoy; however, due to differences in the roles of the CTLA-4/B7 and the PD-1/PD-L pathways, less severe side effects would be seen with PD-1 blockade. Due to the expression patterns of PD-1 and its ligands on both peripheral and immune cells (unlike CTLA-4 and its ligands which are expressed on immune cells only), the paradigm is that PD-1/PD-L pathway predominantly regulates peripheral immune responses at the tissue level (with a lesser role in central immunity than CTLA-4), consistent with its major role in peripheral tolerance. Yervoy (anti-CTLA-4) targets predominantly interactions between T cells and APCs, and is thus expected to cause systemic immune stimulation accompanied by adverse effects. In contrast, due to the large number of studies reporting upregulation of PD-Ls on infected cells and tumor cells, the high-affinity PD-1 Ig would preferentially bind to these target cells and act primarily on T cells (effectors) that are specifically recruited to these sites, as opposed to the systemic immune activation elicited by an antibody that binds to and activates all T cells.

An additional advantage over monoclonal antibodies (such as Yervoy) is the simplicity of making Ig fusion proteins compared to producing monoclonal antibodies. In order to be used in humans, monoclonal antibodies need to be humanized, to prevent secondary immune responses directed against the antibody after repeated treatments. The agents disclosed herein, however, are a modified version of an endogenous human protein, fused to, for example, a human immunoglobulin Fc segment, which makes it fully “human”, minimizing unwanted side-effects associated with sustained treatment. In addition, the agents are able to bind two relevant ligands (PD-L1 and PD-L2), eliminating the need for a combined antibody treatment. Generating recombinant mutant Fc fusion protein is straightforward and less laborious than developing an antibody, which can take longer and be more costly. Notably, the agent disclosed herein is effective as a blocking antibody in binding both PD-ligands and more effective in eliciting activation-induced cytokine responses.

Specific therapeutic targets for the agents disclosed herein are numerous. Both chronic infections and malignant tumors affect increasing numbers of patients not just in the US but worldwide. Chronic infections such as HIV, and HCV affect 33 million and 170 million people worldwide, respectively, as well as HBV infection which affects about 2 billion people worldwide (the most common infectious disease today). Acute infections in which the PD-1 pathway is involved, such as histoplasmosis, are also prevalent (50 million people affected in North America), as are rabies and RSV infections. Malignancies constitute another major area that could be targeted for treatment using the agents of invention. The incidence of tumors continues to increase worldwide; currently about 12.7 million new cases are reported each year. The incidence of cancer cases is highest for Australia/New Zealand, North America and Europe, where treatment is also more available or sought-after. Since several tumor cell types upregulate ligands of inhibitory receptors, such as PD-L1 and PD-L2, to promote immune evasion, administration of the agents disclosed herein is expected to be beneficial in such states.

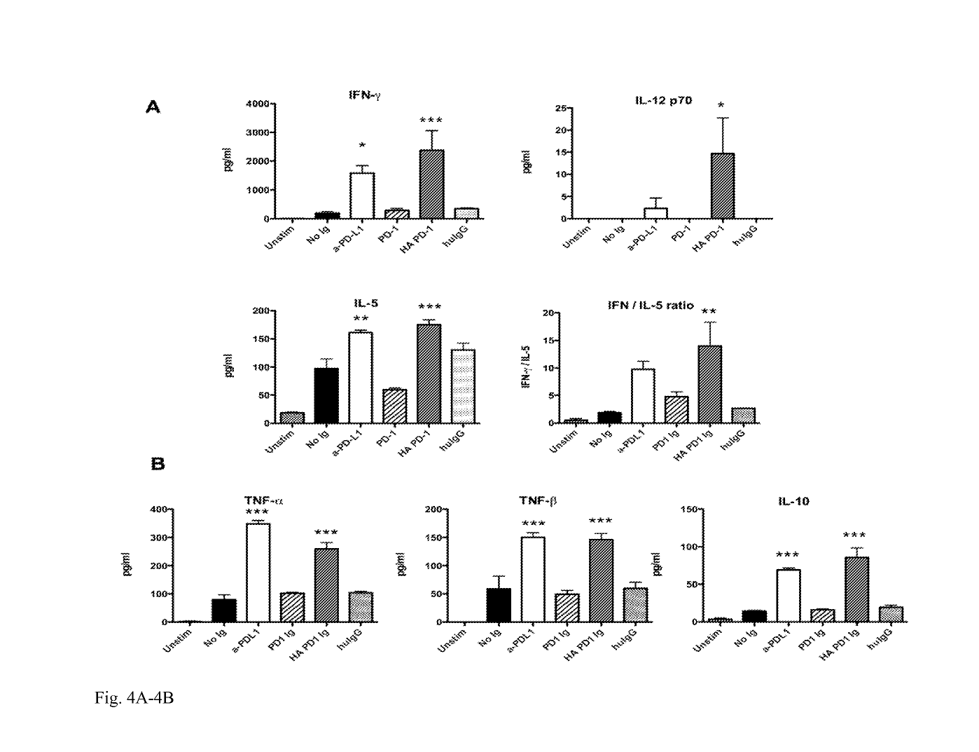

The high-affinity PD-1 Ig (HA PD-1 Ig) was tested using an allogeneic mixed lymphocyte reaction assay (MLR), in which human dendritic cells from one donor were co-cultured with T cells from a different donor, and T cell proliferation and cytokine secretion were determined. When HA PD-1 Ig was added to the co-culture, a significant increase in T cell proliferation was detected compared to wild-type PD-1 Ig or irrelevant huIgG1 control (FIG. 3). Moreover, there were significant increases in the secretion of multiple T cell cytokines after HA PD-1 Ig treatment, the highest increases being detected for interferon-γ and IL-12, but also for IL-5, TNF-α and β and IL-10 (FIG. 4). Of particular note, the effect of the HA PD-1 Ig on human T cell proliferation was comparable to that of a commercial PD-L1 blocking antibody. Thus, by selectively blocking a major T cell inhibitory pathway, the mutant reagent significantly enhances effector T cell responses.

Enhancing T cell activation by interfering with endogenous inhibitory pathways such as PD-1 has enormous potential in the treatment of infectious diseases and malignant tumors. Recent studies have shown that strategies enhancing T cell activation through blockade of PD-1 could be beneficial in chronic viral infections with HIV, HCV, HBV and HTLV. Other diseases in which this co-inhibitory pathway is involved, and in which treatment with the high-affinity PD-1 Ig is expected to be beneficial, include infections with Helicobacter pylori, the fungus Histoplasma capsulatum, parasites such as Taenia crassiceps or Schistosoma mansoni, and the protozoa Leishmania mexicana.

In addition, targeting costimulatory pathways is a promising strategy in tumor therapy. Immunological clearance of tumors is rare, due to the immunosuppressive environment generated by the developing tumor. As part of this immunosuppressive mechanism, many tumors upregulate ligands of inhibitory receptors such as PD-L1 and PD-L2. PD-L1 has been shown to be upregulated on a variety of solid tumors, such as breast, lung, colon, ovarian, melanoma, bladder, liver, salivary, stomach, gliomas, thyroid, thymic, epithelial, head, and neck (Keir M E et al, Annu Rev Immunol 2008). Both PD-Ligands have been shown to be upregulated in hematologic malignancies such as lymphomas and multiple myeloma. In addition, PD-1 is upregulated on tumor infiltrating lymphocytes, which is also expected to contribute to tumor immunosuppression. In addition, HA PD-1 Ig can be beneficial to treat tumors that do not express PD-Ls (although less efficient, compared to PD-L-expressing tumors), by causing immune activation at the level of initial antigen presentation to T cells (an analogous mechanism to that of CTLA4 blockade).

Since the mutant PD-1 Ig is able to bind the PD-Ligands with high affinity, it will prevent inhibitory signals into tumor infiltrating T cells through the endogenous PD-1 receptor. Removal of these inhibitory signals has dramatic effects on anti-tumor immune responses (e.g. as observed with Yervoy, a CTLA-4-specific monoclonal antibody, approved by FDA for the treatment of metastatic melanoma), the fusion polypeptide can be used in the malignancies listed above to activate the tumor-specific T cell response and induce tumor regression.

### REFERENCES

Hodi F S et al., N Engl J Med (2010), 363:711-723.
Keir M E et al, Annu Rev Immunol (2008), 26:677-704

1. An isolated mutant PD-1 polypeptide, wherein the mutant PD-1 polypeptide is a mutant by having an A132L mutation relative to SEQ ID NO:7 or an A132L mutation relative to NCBI Reference Sequence NP_{—}005009.2.

2. The polypeptide of claim 1, wherein the mutant PD-1 comprises consecutive amino acid residues (i) having the sequence set forth in SEQ ID NO:4, or (ii) having a sequence 95% or greater identical to SEQ ID NO:4 and comprising an L at the residue corresponding to A132 relative to NCBI Reference Sequence NP_{—}005009.2.

3. A fusion polypeptide comprising the polypeptide of claim 1 or 2, fused to an immunoglobulin domain polypeptide.

4. The fusion polypeptide of claim 3, wherein the mutant PD-1 polypeptide is fused to the immunoglobulin domain polypeptide by a peptide bond between a terminal amino acid of the mutant PD-1 polypeptide and a terminal amino acid of the immunoglobulin domain polypeptide.

5. The fusion polypeptide of claim 3, wherein the immunoglobulin domain polypeptide comprises an immunoglobulin IgG1 Fc domain.

6. The fusion polypeptide of claim 5, wherein the immunoglobulin IgG1 Fc domain is human.

7. The polypeptide of claim 1.

8. A homo-oligomer comprising the polypeptide of claim 1.

9. A homo-oligomer comprising the fusion polypeptides of claim 3.

10. A composition comprising the polypeptide of claim 1.

11. A composition comprising the fusion polypeptide of claim 3.

12. A composition comprising the homo-oligomer of claim 8.

13. The composition of claim 10, comprising a pharmaceutically acceptable carrier.

14. A method of stimulating T cell activation, treating a tumor, or treating an infection in a subject comprising administering to the subject a composition comprising the polypeptide of claim 1, in an amount sufficient to stimulate T cell activation, treat a tumor, or treat an infection, respectively, in a subject.

15. The method of claim 14, wherein the T cell activation comprises cytokine secretion.

16. The method of claim 14, wherein the subject has a tumor.

17. The method of claim 14, wherein the subject has an infection.

18. An isolated nucleic acid encoding the isolated mutant PD-1 polypeptide of claim 1.

19. An isolated nucleic acid encoding the fusion polypeptide of claim 3.

20. The nucleic acid of claim 18, which is a recombinant nucleic acid.

21-22. (canceled)


SEQUENCE LISTING (7 sequences)

SEQ ID NO: 1 - DNA, 1269 bp, ARTIFICIAL SEQUENCE
  ARTIFICIAL CONSTRUCT BASED ON A HUMAN SEQUENCE
agatctgccgccaccatgtctcgctccgtggccttagctgtgctcgcgctactctctctttctggcctggaggctccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctgcagatcaaagagagcctgcgggcagagctcagggtgacagagagaagggcagaagtgcccacagcccaccccagcccctcacccaggccagccggccagttccaagtcgacttggtccctcgtggtagtggaggctctcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaacatcatcaccatcaccattgaggatcc

SEQ ID NO: 2 - protein, 395 aa, ARTIFICIAL SEQUENCE
  FUSION PROTEIN BASED ON A HUMAN SEQUENCE
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRSAGQFQVDLVPRGSGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 3 - protein, 167 aa, ARTIFICIAL SEQUENCE
  ARTIFICIAL SEQUENCE BASED ON HUMAN SEQUENCE
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQ

SEQ ID NO: 4 - protein, 147 aa, ARTIFICIAL SEQUENCE
  ARTIFICIAL SEQUENCE BASED ON HUMAN SEQUENCE
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQ

SEQ ID NO: 5 - protein, 268 aa, ARTIFICIAL SEQUENCE
  ARTIFICIAL SEQUENCE BASED ON HUMAN SEQUENCE
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 6 - protein, 288 aa, ARTIFICIAL SEQUENCE
  ARTIFICIAL SEQUENCE BASED ON HUMAN SEQUENCE
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 7 - protein, 288 aa, HUMAN
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL